(19) United States
(12) Patent Application Publication
(10) Pub. No.: US 2024/0272162 A1
(43) Pub. Date: Aug. 15, 2024

(54) DRAIN FLUIDS FOR DISEASE DIAGNOSIS AND MONITORING

(71) Applicant: DROPLET BIOSCIENCES, INC., Cambridge, MA (US)

(72) Inventors: Marra S. Francis, Cambridge, MA (US); Jose Zevallos, Cambridge, MA (US); Aadel Chaudhuri, Cambridge, MA (US); Theresa Tribble, Cambridge, MA (US)

(21) Appl. No.: 18/435,187

(22) Filed: Feb. 7, 2024

Related U.S. Application Data
(60) Provisional application No. 63/484,898, filed on Feb. 14, 2023.

(51) Int. Cl. G01N 33/574; C12Q 1/6886

(52) U.S. Cl. CPC G01N 33/57488; C12Q 1/6886; C12Q 2600/156; G01N 2333/525; G01N 2333/5412; G01N 2333/5428; G01N 2333/545; G01N 2333/96494

(57) ABSTRACT

The present invention provides methods for using effluent obtained from medical procedures resulting in stomas or percutaneous drains or ports to assess diagnostic biomarkers indicative of disease. Identified biomarkers are used to inform surgical success and/or to assess and monitor efficacy of treatment.

### TECHNICAL FIELD

The invention relates to diagnostic methods for identifying biomarkers indicative of disease.

### BACKGROUND

Cancer is a leading cause of death. Early detection, while beneficial for most cancers, is often difficult. In part, this is because many cancers first develop without presenting any specific clinical symptoms, and diagnosis only occurs when the disease has reached a stage when it is difficult to treat. Many cancers, such as bladder, colon, small intestine, and kidney cancers, require invasive procedures for definitive diagnosis. Further, some cancers require repeated invasive procedures to monitor for disease recurrence. Tissue, such as tumor tissue, generally is the most informative sample for diagnosis and prognosis of cancer. Pathology is typically performed on samples obtained from the primary lesion (e.g., a solid tumor) or from a lymph gland (e.g., a sentinel lymph gland). Unfortunately, tissue samples are often difficult to access and subject to limited availability, especially without performing an invasive procedure. In the context of cancer, often by the time tumors are detected, cancer has spread or progressed.

Conventional cancer detection and monitoring has focused on liquid biopsy in blood or plasma for the detection of cell-free tumor DNA. Blood is of high clinical interest because of its accessibility. Unfortunately, many of these methods lack sensitivity. As a result, early cancer detection, when tumor DNA is present as only a minute fraction of the DNA collected from blood or plasma, is often difficult. Moreover, due to the lack of sensitivity, progression of the disease and its response to therapeutic intervention are difficult to monitor.

Cancer-related surgical interventions resulting in stomas or percutaneous drains or ports give rise to the expression of effluent or drain fluid regarded as waste. Other than assessing for evidence of infection, which usually involves pus and other detritus from bacterial cells, effluent is generally considered waste and is not used for diagnostic or monitoring purposes. The invention described below provides an alternative source of diagnostic information that leads to greater precision in informing surgical success, and disease diagnosis, monitoring, and treatment.

### SUMMARY

The present invention provides methods for the assessment of diagnostic biomarkers in effluent obtained from medical procedures resulting in stomas or percutaneous drains or ports. For example, an ostomy is a surgical procedure that involves the removal of diseased portions of the gastrointestinal or urinary system and creation of an artificial opening (stoma) in the abdomen to allow for the elimination of body wastes. An intraperitoneal drain is a tube placed in the abdomen to remove fluid from the intraperitoneal space. Other than assessing the effluent for evidence of infection, which usually involves pus and other detritus from bacterial cells, drainage effluent from these types of surgical interventions is generally considered waste, is removed, either passively or actively, and discarded. The effluent is typically not used for diagnostic or monitoring purposes. According to the invention, this effluent is actually a rich source of diagnostic information.

Sample used in methods of the invention may be collected from a colostomy, ileostomy, or urostomy pouch or bag, a percutaneous catheter, peritoneal port, needle aspiration (e.g., from a lumpectomy or other procedure in which effluent is drawn from within the body), intracranial drain, lumbar drain UP shunt, or intraperitoneal drain. The sample may also be collected through paracentesis in assessing ascites. Thus, the invention provides for using effluent as a source of biomarkers indicative of disease to assess disease state, including presence or absence of disease, minimal residual disease, recurrence, and therapeutic efficacy. Methods taught herein provide non-invasive, sensitive, and specific diagnostics that allow assessment of disease status, progression, and therapeutic efficacy.

In one aspect, the present invention provides methods for assessing disease state by obtaining effluent from a percutaneous catheter, peritoneal drain or an ostomy or anastomosis and identifying biomarkers indicative of disease state in the effluent. The effluent may comprise, for example, fecal matter, mucus, urine, bile, blood, plasma, aggregated tissue, irrigation fluid, lymphatic fluid, lymphovascular fluid, interstitial fluid, cells, cellular debris, bacteria, protein, and nucleic acid, or a combination thereof. The fluid may further comprise sweat, semen, vaginal secretions, cerebrospinal fluid, synovial fluid, pleural fluid, peritoneal fluid, pericardial fluid, amniotic fluid, or saliva depending on the location from which it is obtained. Accordingly, it may be necessary to isolate a fraction of interest from the effluent. However, the invention contemplates that raw effluent contains sufficient diagnostic content that it can be used without any significant sample preparation.

In preferred embodiments, the sample is obtained from a bag or pouch system connected to a stoma created by an ostomy. For example, the sample is collected from a colostomy, ileostomy, or urostomy bag or pouch. In other embodiments, the sample is collected from a percutaneous catheter, needle, peritoneal port, or intraperitoneal drain.

In various embodiments, the presence of certain biomarkers are indicative of minimal residual disease. Effluent may be collected at or near a site of clinical significance, e.g., a site of a resected tumor. Therefore, biomarkers found in the effluent are useful to report on the local environment associated with a disease. Biomarkers can reveal residual disease as well as factors that led to disease formation, e.g., tumor growth, which is useful to assess the risk of disease recurrence.

Biomarkers collected from effluent are also informative of immune and/or inflammatory response following an interventional procedure. Quantities of certain biomarkers, and combinations thereof, are also correlated with known clinical outcomes to determine disease prognosis. Accordingly, methods of the invention use biomarkers collected from effluent to detect minimal residual disease or determine the likelihood of recurrence.

Biomarkers are selected from a nucleic acid, protein, bacteria, cells, and viruses. In preferred embodiments, the cell is a cancer cell. However, biomarkers for use in the invention may be any known biomarker for disease. For example, the biomarker may be a nucleic acid (DNA, any RNA species), a protein, or any other molecule or compound. The origin of the biomarker may be an organism, for example a bacterium, a fungus, an animal cell, a tumor cell, a protozoan cell, or a virus.

The present invention is useful for evaluation of any disease biomarker. In a preferred embodiment, effluent is a source of circulating tumor DNA (ctDNA), tumor cells, ratios of ctDNA to cells, mutations associated with cancer, oncogenes and the like. The invention is also useful for the assessment of diseases other than cancer, for example, infectious diseases, autoimmune diseases, and endocrine diseases. However, the invention has particular application in the diagnosis, monitoring therapeutic efficacy and treatment of cancer.

Biomarkers are correlated with the diagnosis, prognosis, staging and development of disease, as well as therapeutic selection and response. Biomarkers collected from the sample are useful to diagnosis disease and to assess disease severity. For example, quantities of certain biomarkers, or combinations of biomarkers, identified may be correlated with a known clinical outcome or a treatment response. As such, methods of the invention are useful to identify an optimal treatment for a patient or identify a treatment, such as chemotherapy, that can be avoided.

In clinical oncology, early diagnosis and prognosis is critical for appropriate therapeutic intervention. For example, treatment protocols may be different if there are indications of metastatic disease in cancer. Accordingly, biomarkers detected in the sample may depend on the purpose for the assessment, e.g., to indicate normal biological processes, pathogenic processes, or pharmacologic response to therapeutic intervention. Therefore, the choice of specific biomarkers is dependent on the sensitivity and specificity of the biomarker for a particular condition, and also on technical aspects related to collection protocols, stability and detection from biological samples.

In certain embodiments, the invention provides methods for assessing surgical success. Post-surgical effluent obtained from a stoma is assessed for the presence of biomarker indicative of the success or failure of the surgery. In one example, the biomarker is tumor DNA or RNA, the presence of which is indicative of the presence of tumor post-resection.

Similarly, the presence of disease-associated biomarkers in effluent is useful to determine local or systemic spread. For example, a DNA sample from a tumor may be obtained as well as a blood sample and a sample of effluent. Each of the samples may then be tested for tumor DNA, and the ratios of tumor DNA in the tumor, effluent sample, and blood are used as an indicator of metastasis.

Methods of the invention also contemplate assessment of pharmaceutical efficacy. According to the invention, accumulation of tumor DNA in effluent after therapy is indicative that the therapy is effective. An increase in tumor DNA in effluent after therapy is indicative of the induction of cell death in the tumor. Thus, non-invasive, real-time measurement during therapy is indicative of therapeutic efficacy. Thus, the efficacy of systemic therapy is measured by the concentration of local biomarkers in the effluent. These methods are agnostic in terms of biomarker content.

Some embodiments of the invention include separating lymphatic fluid, or components thereof, from the sample of effluent. According to aspects of the invention, the separated portion of the sample will contain a greater quantity of biomarkers than can be obtained from an equal volume of blood.

In another aspect, the invention provides for a method for monitoring the efficacy of a treatment, comprising the steps of obtaining, as a first sample, effluent from a patient having undergone a surgical procedure, wherein the first sample is obtained at a first time point; obtaining, as a second sample, effluent from the patient, wherein the second sample is obtained at a second timepoint; identifying in the sample a difference in the first sample and the second sample in a biomarker indicative of disease; and determining the efficacy of treatment based on the difference.

In embodiments, the sample is obtained at or near the time of the surgical procedure at a first time point. The method contemplates obtaining a second sample at a second time point identifying in the sample a difference in a biomarker(s) or the presence or absence of the biomarker indicative of disease. The presence or absence of the biomarker, or the identified difference, is compared. For samples that have been obtained after the patient has undergone treatment, the comparison of samples is an indication treatment efficacy.

In preferred embodiments, the sample is obtained from a bag or pouch system connected to a stoma created by an ostomy. For example, the sample is collected from a colostomy, ileostomy, or urostomy bag or pouch. In other embodiments, the sample is collected from a percutaneous catheter, needle, peritoneal port, or intraperitoneal drain. Thus, when the surgical procedure is related to cancer treatment, methods of the invention provide a means for noninvasive cancer management by evaluating waste and by-products collected from sites related to diseased or wounded tissue. The by-products may contain material informative of an excised tumor as well as the milieu that surrounded the tumor providing insights into the physiological conditions that gave rise to the tumor.

In certain embodiments, the presence of the biomarker is indicative of minimal residual disease. Biomarkers collected from effluent can inform on a patient's immune and/or inflammatory response following a tumor resection. Presence or absence of specific biomarkers correlate to the level of efficacy of treatment. Quantities of certain biomarkers, and combinations thereof, can also be correlated with known patient outcomes to determine a disease prognosis. Accordingly, methods of the invention can use biomarkers collected from effluent to detect minimal residual disease or determine whether the disease is likely to recur.

The biomarker may be selected from a nucleic acid, a protein, a bacterium, a cell, and a virus. In preferred embodiments, the cell is a cancer cell. However, biomarkers for use in the invention may be any known biomarker for disease. For example, the biomarker may be a nucleic acid (DNA, any RNA species), a protein, or any other molecule or compound. The origin of the biomarker may be an organism, for example a bacterium, a fungus, an animal cell, a tumor cell, a protozoan cell, or a virus. In a preferred embodiment, effluent is a source of circulating tumor DNA (ctDNA), tumor cells, ratios of ctDNA to cells, mutations associated with cancer, oncogenes and the like. The invention is also useful for the assessment of diseases other than cancer, for example, infectious diseases, autoimmune diseases, or endocrine diseases. However, the invention has particular application in the diagnosis, monitoring and treatment of cancer.

In embodiments, the invention contemplates that the biomarker is a ratio of circulating tumor cells to cell-free DNA. The biomarker may be compared to an amount determined in blood or lymph node. In embodiments, the biomarker comprises one or more of interleukin-1, interleukin-6, interleukin-10, a tumor necrosis factor, matrix metalloproteinase-1, matrix metalloproteinase-2, matrix metalloproteinase-9, matrix metalloproteinase-13, or a nucleic acid comprising a mutation. The method may further comprise the step of obtaining a genomic profile from the sample.

Similarly, the presence of disease-associated biomarkers in effluent is useful to determine systemic or local spread and disease progression based on the presence or absence of the biomarker in the sample of effluent. For example, a DNA sample from a tumor may be obtained as well as a blood sample and effluent. Each of the samples may then be tested for tumor DNA, and the ratios of tumor DNA in the tumor, effluent, and blood may be used as an indicator of disease staging. In embodiments, the methods of the invention contemplate diagnosing disease based on the presence or a biomarker in the sample.

### DETAILED DESCRIPTION

The invention described provides an alternative source of diagnostic information that leads to greater precision in informing surgical success, and disease diagnosis, monitoring, and treatment. In embodiments, the invention is applicable to effluent obtained as part of a surgical procedure. The scope of the invention is not limited to any one type of surgery. The surgery can be any form of bodily intervention, including an intervention that is wholly unrelated to disease. In some embodiments, the surgery is a resection surgery or anastomosis.

In specific applications, the invention provides methods for using effluent obtained from medical procedures resulting in stomas or percutaneous drains or ports to assess diagnostic biomarkers indicative of disease.

As an example, an ostomy is a surgical procedure that involves the removal of diseased portions of the gastrointestinal or urinary system and creation of an artificial opening (stoma) in the abdomen to allow for the elimination of body wastes. An intraperitoneal drain is a tube placed in the abdomen to remove fluid from the intraperitoneal space. The surgical procedure may be paracentesis for assessing ascites. Other than assessing the effluent for evidence of infection, which usually involves pus and other detritus from bacterial cells, drainage effluent from these types of surgical interventions is generally considered waste and is not used for diagnostic or monitoring purposes.

In one aspect, the invention provides a method for assessing surgical success. The method may comprise the steps of obtaining, as a sample, effluent from a patient having undergone a surgical procedure; identifying in the sample the presence or absence of a biomarker indicative of disease; and determining the success of the surgery based on the presence or absence of the biomarker in the sample.

The surgical procedure may be any procedure. In non-limiting examples, the surgery may be for ovarian, stomach, small intestine, colorectal, rectal, anal, bladder or prostate cancer. The surgery may be radical cystectomy. The surgery may be anastomosis. The waste fluid may or may not be in proximity to the surgical site. There are many types of surgeries (i.e. ostomy) resulting in a stoma, and intestinal stomas are necessary for several colon and rectal conditions such as colorectal cancer, bladder cancer, Crohn's disease, ulcerative colitis, birth defects, and other intestinal or urinary medical conditions. Any hollow organ can be manipulated into an artificial stoma as necessary, including the esophagus, stomach, duodenum, ileum, colon, pleural cavity, ureters, urinary bladder, and renal pelvis.

An ostomy involves the removal of diseased portions of the gastrointestinal or urinary system and creation of an artificial opening in the abdomen to allow for the elimination of body wastes. Common surgeries include colostomy, ileostomy, jejunostomy, duodenostomy, gastrostomy, and urostomy. A colostomy is an opening in the large intestine (colon), or the surgical procedure that creates one. The opening is formed by drawing the healthy end of the colon through an incision in the anterior abdominal wall and suturing it into place. This opening, often in conjunction with an attached ostomy system, provides an alternative channel for feces to leave the body. Thus, if the natural anus is unavailable for that function (for example, in cases where it has been removed because of colorectal cancer or ulcerative colitis), an artificial anus takes over. In an ileostomy, the bottom of the small intestine (ileum) is attached to the stoma to bypass the colon, rectum, and anus. In a urostomy, tubes that carry urine to the bladder are attached to the stoma to bypass the bladder. Ileoanal pouch surgery is another type of bowel surgery that usually requires a temporary ileostomy.

A pouching or bag system may collect the output (effluent) from a colostomy, ileostomy or urostomy. In embodiments of the invention, the sample is obtained or collected from the pouching or bag system associated with the ostomy. Specifically, the sample may be collected from a colostomy, ileostomy, or urostomy bag or pouch.

In some embodiments, the surgical procedure may be a surgical anastomosis wherein a segment of intestine, blood vessel, or other structure is connected together. Common surgical anastomoses include intestinal anastomosis, Roux-en-Y anastomosis, or ureteroureterostomy. It is contemplated that methods of the invention may be used to monitor for anastomotic leak, a condition in which the anastomosis fails and contents of a reconnected body channel leak from the surgical connection. An intraperitoneal drain may be placed into the abdomen to remove fluids from the intraperitoneal space. In embodiments of the invention, the sample is obtained or collected from the drain, needle, catheter, or port. In specific embodiments, the sample may be collected from a percutaneous catheter, peritoneal port, or intraperitoneal drain. The sample may be collected through paracentesis in assessing ascites. Ascites is a build up of fluids in the abdomen, often as a result of severe liver disease.

Because the sample may be obtained from a bag or pouch system associated with an ostomy, or other catheter, drain, needle, port, or collection vessel, the invention contemplates that the sample obtained may be conventionally considered waste. For example, the sample may be effluent comprising, for example, feces, mucus, urine, bile, blood, plasma, peritoneal fluid, aggregated tissue, irrigation fluid, lymphatic fluid, lymphovascular fluid, interstitial fluid, cells, cellular debris, bacteria, protein, and nucleic acid, or a combination thereof. The sample may further comprise sweat, semen, vaginal secretions, cerebrospinal fluid, synovial fluid, pleural fluid, peritoneal fluid, pericardial fluid, amniotic fluid, or saliva depending on the location from which it is obtained.

The biomarker identified by methods of the present invention may be any known biomarker for a given disease present in the effluent. Biomarkers useful in the invention vary and are selected based on the disease indication being monitored and other factors known to the skilled artisan. Moreover, sensitivity and specificity may vary across biomarkers and that will influence biomarker selection.

For example, the biomarker may comprise tumor cells, immune cells, bacterial cells, viral host cells, donor organ cells, microvascular cells, cell-free DNA, cell-free RNA, circulating tumor DNA, messenger RNA, miRNA, exosomes, proteins, hormones, and other analytes. The biomarker identified may depend on, for example, a specific patient, pathology, surgery type, and surgery site. By analyzing biomarkers in the sample, methods of the invention may provide diagnostic or prognostic information. For example, by identifying circulating tumor cells or cell-free tumor DNA, cancer may be diagnosed in the subject.

In various aspects, biomarkers may be identified and quantified using methods known in the art. Suitable assays include, for example, nucleic acid sequencing, PCR, quantitative PCR, digital droplet PCR, Western blot target capture, proteomics, nucleic acid expression analysis, and antibody screening. For example, assays may include whole genome sequencing, next generation DNA sequencing, next generation RNA sequencing, multiplex PCR, methylation analysis, droplet PCR, droplet cell separation, or any combination thereof.

Fluorescent labels may be used to identify biomarkers. A fluorescent label or fluorescent probe is a molecule that is attached chemically to aid in the detection of a biomarker. Fluorescent labeling generally uses a reactive derivative of a fluorescent molecule known as a fluorophore. The fluorophore selectively binds to a specific region or functional group on the biomarker and can be attached chemically or biologically. Any known technique for fluorescent labeling may be used, for example enzymatic labeling, protein labeling, or genetic labeling. Any known fluorophore may also be used. Both the fluorophore and labelling technique may be selected and adjusted based on the biomarker to be identified. The most commonly labelled molecules are antibodies, proteins, amino acids and peptides which are then used as specific probes for detection of a particular target.

Fluorescent labelling may be used to identify and quantify a biomarker in the effluent sample without separating the components of the surgical fluid. For example, by providing fluorescent labels directly into the sample, fluorescent microscopy or a colorimetric assay can be used to identify and quantify the presence of the biomarker from a color change alone.

When quantifying a biomarker, barcodes may be added to the biomarker to aid in amplification, detection, or differentiation of the biomarker. Barcodes may be added to biomarkers by “tagging” the biomarker with the barcode. Tagging may be performed using any known method for barcode addition, for example direct ligation of barcodes to one or more of the ends of a nucleic acid molecule or protein. Nucleic acid molecules may, for example, be end repaired in order to allow for direct or blunt-ended ligation of the barcodes. Barcodes may also be added to nucleic acid molecules through first or second strand synthesis, for example using capture probes or primers. First and second strand synthesis is advantageously used in RNA analysis to generate tagged DNA molecules.

Unique molecular identifiers (UMIs) are a type of barcode that may be provided to biomarkers in a sample to make each biomarker, together with its barcode, unique, or nearly unique. For example, with regard to nucleic acid molecules, this is accomplished by adding, e.g. by ligation or reverse transcription, one or more UMIs to each nucleic acid molecule such that it is unlikely that any two previously identical nucleic acid molecules, together with their UMIs, have the same sequence. By selecting an appropriate number of UMIs, every nucleic acid molecule in the sample, together with its UMI, will be unique or nearly unique. One strategy for doing so is to provide to a sample of nucleic acid molecules a number of UMIs in excess of the number of starting nucleic acid molecules in the sample. Thus, each starting nucleic molecule will be provided with different UMIs, therefore making each molecule together with its UMIs unique. However, the number of UMIs provided may be as few as the number of identical nucleic acid molecules in the original sample. For example, where no more than six nucleic acid molecules in a sample are likely to be identical, as few as six different UMIs may be provided, regardless of the number of starting nucleic acid molecules.

UMIs are also advantageous in that they can be useful to correct for errors created during amplification, such as amplification bias or incorrect base pairing during amplification. For example, when using UMIs, because every nucleic acid molecule in a sample together with its UMI or UMIs is unique or nearly unique, after amplification and sequencing, molecules with identical sequences may be considered to refer to the same starting nucleic acid molecule, thereby reducing amplification bias. Methods for error correction using UMIs are described in Karlsson et al., 2016, “Counting Molecules in cell-free DNA and single cells RNA”, Karolinska Institutet, Stockholm Sweden, the contests of which are incorporated herein by reference.

For RNA or mRNA sequencing, sequencing may first comprise the step of preparing a cDNA library from barcoded RNA, for example through reverse transcription, and sequencing the cDNA. cDNA sequencing may advantageously allow for the quantification of gene expression within the single cell, and can be useful to identify characteristics of the single cell to, for example, make a diagnosis, prognosis, or determine drug effectiveness.

Reverse transcription may be performed using without limitation dNTPs (mix of the nucleotides dATP, dCTP, dGTP and dTTP), buffer/s, detergent/s, or solvent/s, as required, and suitable enzyme such as polymerase or reverse transcriptase. The polymerase used may be a DNA polymerase, and may be selected from Taq DNA polymerase, Phusion polymerase (as provided by Thermo Fisher Scientific, Waltham, Massachusetts), or Q5 polymerase. Nucleic acid amplification reagents are commercially available, and may be purchased from, for example, New England Biolabs, Ipswich, MA, USA. The reverse transcriptase used in the presently disclosed targeted library preparation method may be for example, maxima reverse transcriptase. In some embodiments, the general parameters of the reverse transcription reaction comprise an incubation of about 15 minutes at 25 degrees and a subsequent incubation of about 90 minutes at 52 degrees.

Reverse transcription may be performed by oligos that have a free, 3′ poly-T region. The 3′ portions of the cDNA capture oligos may include gene-specific sequences or oligomers, for example capture primers to reverse transcribe RNA guides comprising a capture sequence. The oligomers may be random or “not-so-random” (NSR) oligomers (NSROs), such as random hexamers or NSR hexamers. The oligos may include one or more handles such as primer binding sequences cognate to PCR primers that are used in the amplifying step or the sequences of NGS sequencing adaptors. The reverse transcription primers may include template switching oligos (TSOs), which may include poly-G sequences that hybridize to and capture poly-C segments added during reverse transcription.

Reverse transcription of non-polyadenylated RNA may comprise use of a capture sequence and a capture primer or probe. Primer sequences may comprise a binding site, for example a primer sequence that would be expected to hybridize to a complementary sequence, if present, on any nucleic acid molecule released from a cell and provide an initiation site for a reaction. The primer sequence may also be a “universal” primer sequence, i.e. a sequence that is complementary to nucleotide sequences that are very common for a particular set of nucleic acid fragments. Primer sequences may be P5 and P7 primers as provided by Illumina, Inc., San Diego, California. The primer sequence may also allow a capture probe to bind to a solid support.

Reverse transcription can also be useful for adding a barcode or a UMI, or both to cDNA. This process may comprise hybridizing the reverse transcription primer to the probe followed by a reverse transcription reaction. The complement of a nucleic acid when aligned need not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

Nucleic acid molecules may advantageously be amplified prior to sequencing. Amplification may comprise methods for creating copies of nucleic acids by using thermal cycling to expose reactants to repeated cycles of heating and cooling, and to permit different temperature-dependent reactions (e.g. by Polymerase chain reaction (PCR). Any suitable PCR method known in the art may be used in connection with the presently described methods. Non limiting examples of PCR reactions include real-time PCR, nested PCR, multiplex PCR, quantitative PCR, or touchdown PCR.

Sequencing nucleic acid molecules may be performed by methods known in the art. For example, see, generally, Quail, et al., 2012, A tale of three next generation sequencing platforms: comparison of Ion Torrent, Pacific Biosciences and Illumina MiSeq sequencers, BMC Genomics 13:341. Nucleic acid molecule sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

The conventional pipeline for processing sequencing data includes generating FASTQ-format files that contain reads sequenced from a next generation sequencing platform, aligning these reads to an annotated reference genome, and quantifying expression of genes. These steps are routinely performed using known computer algorithms, which a person skilled in the art will recognize can be used for executing steps of the present invention. For example, see Kukurba, Cold Spring Harb Protoc, 2015 (11):951-969, incorporated by reference.

The method provides for determining success of a surgery based on the presence or absence of biomarkers identified in the sample. Twenty-five percent of stage II colon cancer patients relapse within 5 years due to minimal residual disease (MRD) not eliminated by surgery. In a non-limiting example, a patient may undergo a bowel resection surgery or colectomy to remove cancerous tumors, which results in an ostomy. The effluent collected in the colostomy bag is regarded as waste. However, according to methods of the invention, this effluent is rich in diagnostic material. The biomarker of interest may be, for example, circulating tumor cells, or circulating tumor-derived factors such as cell-free tumor DNA (ctDNA). ctDNA may indicate the presence of MRD following surgical resection and predict risk of recurrence with a high degree of precision.

Thus, a sample obtained from this effluent may be used to identify the presence or absence of a biomarker related to the cancer removed from the patient. Evidence of a positive biomarker in the sample may indicate that not all of the tumor was removed by the surgery. Alternatively, in embodiments, the biomarker identified in the sample is indicative of minimal residual disease.

MRD can be defined as cancer persisting in a patient, after treatment, that may not be detected with current medical imaging modalities and is, therefore, an occult stage of cancer progression. Methods of the invention use biomarkers from effluent to evaluate MRD to see if the cancer treatment is working and to guide further treatment plans. Methods of the invention use liquid biopsy approaches based on the detection of small numbers of circulating tumor cells (CTCs) or minute amounts of circulating cell-free tumor DNA (ctDNA) to detect MRD in patients with various malignancies. Using methods of the invention, monitoring CTCs and ctDNA during post-surgical follow-up assessments enables detection of disease relapse, or determines the success of surgical outcomes, earlier than is possible with current radiological imaging procedures. Further characterization of CTCs and ctDNA provides insights into the molecular evolution of MRD during tumor progression with implications for therapeutic interventions to delay or prevent metastatic relapse.

Methods of the invention may use utDNA, cfDNA, or ctDNA as biomarkers for surgical success, disease recurrence, and/or determining the efficacy of treatment. As an example, urinary tract DNA (utDNA) and ctDNA are associated with the progression from non-muscle-invasive to muscle-invasive disease. ctDNA is associated with a worse overall survival and disease recurrence. Methods of the invention may use the sample from effluent to monitor for ctDNA and indications of cancer recurrence. As another example, cell-free DNA (cfDNA) is significantly higher in patients with kidney cancer than in healthy controls or patients with benign lesions. Detected ctDNA and increased cfDNA are associated with decreased survival. Thus, methods of the invention can detect cfDNA that may be associated with kidney cancer.

In another example, the measurement of biomarkers in peritoneal fluid such as cytokines or matrix metalloproteinases in the early post-operative period helps diagnose anastomotic leakage at a preclinical stage. Anastomotic leak in colorectal surgery leads to significant morbidity, mortality and poorer oncological outcomes. Diagnosis of anastomotic leak is frequently delayed as current methods of detection are not sensitive or specific enough. Methods of the invention provide for detection of biomarkers such as cytokines or matrix metalloproteinases in early post-operative periods after anastomosis. Thus, methods of the invention not only provide for determining the success of a surgery but also allow for monitoring for disease recurrence, and the efficacy of treatment.

In certain embodiments, the biomarker may be selected from a nucleic acid, a protein, a bacterium, a cell, and a virus. As discussed above, the cell may be a cancer cell. Contemplated methods discussed below comprise identifying circulating tumor cells in lymphatic fluid at two or more time points (i.e., serial monitoring); identifying cell-free tumor DNA in the sample of effluent at two or more time points; comparing a ratio of circulating tumor cells to cell-free DNA at the same two or more time points; and assessing the progression of disease or the efficacy of treatment as a changing ratio of cell-free tumor DNA to circulating tumor cells over the time interval of the two time points.

In other embodiments, the biomarker may be compared to an amount determined in blood or lymph node. In embodiments, the biomarker comprises one or more of interleukin-1, interleukin-6, interleukin-10, a tumor necrosis factor, matrix metalloproteinase-1, matrix metalloproteinase-2, matrix metalloproteinase-9, matrix metalloproteinase-13, or a nucleic acid comprising a mutation. The method may further comprise the step of obtaining a genomic profile from the sample. For example, biomarkers for disease may be detected by, for example, protein or nucleic acid sequencing. The biomarker may be DNA and/or RNA. Accordingly, methods of the present invention contemplate creating a genomic profile from DNA or RNA in the drain fluid. The profile may be a patient's germ line.

In another aspect, the invention provides a method of monitoring efficacy of a treatment, the method comprising the steps of: obtaining, as a first sample, effluent from a patient having undergone a surgical procedure, wherein the first sample is obtained at a first time point; obtaining, as a second sample, effluent from the patient, wherein the second sample is obtained at a second timepoint; identifying in the sample a difference in the first sample and the second sample in a biomarker indicative of disease; and determining the efficacy of treatment based on the difference.

The first sample may be obtained during or near the time of the surgical procedure. The second or multiple subsequent samples may be obtained while the patient is undergoing a separate, surgical procedure occurring at a different time point than the first surgical procedure. In embodiments, the first, second, or subsequent samples are obtained from a bag or pouch system associated with an ostomy, a percutaneous catheter, peritoneal port, needle, or intraperitoneal drain. The collection times are specific points in time wherein the second fluid sample is taken at a later point in time than the first fluid sample. In embodiments, the second sample, or subsequent samples, is taken post-treatment. A minimum of a first and second sample at first and second time points are collected. However, multiple subsequent samples at corresponding multiple subsequent timepoints may also be collected.

Methods of the invention comprise identifying a difference, or differences, between the first sample and the second sample. Treatment efficacy, as well as assessing disease progression, severity, staging, prognosis or diagnosis may be evaluated based on the difference or differences. Identified differences include presence or absence of one or more biomarkers, changes in quantity, amount, weighted amount, quality, heterogeneity (both in terms of genomic heterogeneity and morphologic heterogeneity), velocity of change, and/or accumulated changes over time between two or more measurements. For example, differences may include the rate of accumulation of a biomarker, an amount of tumor cells, or an amount of cell-free DNA or RNA. The difference may also be the fragment size of cell-free DNA or RNA wherein a decrease in average fragment size between the first effluent sample at the first time point and the second effluent sample at a second time points is indicative of disease progression. The difference may also be measured as presence/absence or positive/negative for the presence of a biomarker or set of biomarkers.

If a biomarker or other quantity of interest has increased in concentration between the first sample at the first time point, and the second sample at the second time point, a difference is identified which is an indication of disease progression or advancement, or that treatment is not efficacious. For example, in cancer, disease progression is often defined by cancer that continues to grow or spread. Progression-free survival (PFS) for patients with cancer is the length of time during and after treatment of a disease that a patient lives with the disease while the disease does not worsen. For clinical trials, measuring the progression-free survival (PFS) is one way to see how well a new treatment works. Therefore, the information obtained from the difference identified between the first sample and the second sample may be used to assess disease progression and treatment efficacy. Thus, methods of the invention are useful to identify effective therapeutics and to assess the efficacy of treatments.

In one embodiment, methods of the invention assess a rate of accumulation of a biomarker as indicative of disease status or progression. The rate of accumulation of the biomarker may be represented as the gradual acquisition of a mass or quantity over time, or the progressive increase in concentration over time. According to methods of the invention the rate of accumulation or decrease of biomarkers in a sample of effluent may be indicative of disease severity and whether a disease is progressing or regressing. For example, if the effluent is measured at multiple time points, it is possible to calculate a slope of biomarker accumulation. The steepness of the slope is indicative of the velocity of change (either negative or positive). In the same way, the area under the curve resulting from multiple measurements in the drain fluid is indicative of disease progression or regression. In assessing disease progression, the rate of accumulation may offer information about the effect of therapeutic intervention. In addition, the rate of accumulation may identify different tumor types that are amenable to certain therapies and may also identify patients who will benefit from such treatment. The concentration, mass or quantity of a biomarker in the first sample and again in the second or subsequent samples may be measured. The difference in concentration, mass or quantity of a biomarker or plurality of biomarkers over time is used to calculate the rate of accumulation of the biomarker.

As noted above, in embodiments, assessing efficacy of treatment or disease progression may be evaluated by identifying the difference in amounts of tumor cells in first and second samples. The exact nature of the cell being measured may vary. For example, tumor cells may be, for example, circulating tumor cells, tumor-derived exosomes, or circulating tumor nucleic acids. The invention also contemplates detecting circulating tumor nucleic acids released from tumor cells. Tumor cells in the sample are quantified using any suitable detection technologies with or without enrichment, including but not limited to, fluorescence, surface-enhanced Raman scattering, or electrical impedance. The invention provides for assessing disease progression by identifying differences in an amount of tumor cells in first and subsequent effluent samples. Differences may be used to monitor disease progression, diagnosis, chemotherapeutic efficacy, and may also provide insight into the biology of metastatic cancer.

As discussed above, the surgical procedure may be any procedure. In non-limiting examples, the surgery may be for colorectal, small intestine, ovarian, rectal, anal, bladder or prostate cancer (or any other cancer). The surgery may be radical cystectomy. The surgery may be anastomosis. The waste fluid may or may not be in the proximity to the surgical site. There are many types of surgeries (i.e. ostomy) resulting in a stoma, and intestinal stomas are necessary for several colon and rectal conditions such as colorectal cancer, bladder cancer, Crohn's disease, ulcerative colitis, birth defects, and other intestinal or urinary medical conditions. Any hollow organ can be manipulated into an artificial stoma as necessary, including the esophagus, stomach, duodenum, ileum, colon, pleural cavity, ureters, urinary bladder, and renal pelvis.

Also discussed above, an ostomy is a surgical procedure that involves the removal of diseased portions of the gastrointestinal or urinary system and creation of an artificial opening in the abdomen to allow for the elimination of body wastes. In embodiments of the invention, the sample is obtained or collected from the pouching or bag system associated with an ostomy. Specifically, the sample may be collected from a colostomy, ileostomy, or urostomy bag or pouch.

In some embodiments, the surgical procedure may be a surgical anastomosis wherein a segment of intestine, blood vessel, or other structure is connected together. Common surgical anastomoses include intestinal anastomosis, Roux-en-Y anastomosis, or ureteroureterostomy. It is contemplated that methods of the invention may be used to monitor for anastomotic leak, a condition in which the anastomosis fails and contents of a reconnected body channel leak from the surgical connection. An intraperitoneal drain may be placed into the abdomen to remove fluids from the intraperitoneal space. In embodiments of the invention, the sample is obtained or collected from the drain, needle, catheter, or port associated with an anastomosis. In specific embodiments, the sample may be collected from a percutaneous catheter, needle, peritoneal port, or intraperitoneal drain. In embodiments, the sample may be collected through paracentesis in assessing ascites.

The sample is obtained from a patient having undergone a surgical procedure. In embodiments, the sample is obtained from a bag or pouch system associated with an ostomy, or other catheter, drain, port, or collection vessel. The invention contemplates that the sample obtained may be conventionally considered waste. For example, the sample may be effluent comprising, for example, feces, mucus, urine, bile, blood, plasma, peritoneal fluid, aggregated tissue, irrigation fluid, lymphatic fluid, lymphovascular fluid, interstitial fluid, cells, cellular debris, bacteria, protein, and nucleic acid, or a combination thereof. The sample may further comprise sweat, semen, vaginal secretions, cerebrospinal fluid, synovial fluid, pleural fluid, peritoneal fluid, pericardial fluid, amniotic fluid, or saliva depending on the location from which it is obtained.

The biomarker identified by the present invention may be any known biomarker for a given disease present in the effluent. Biomarkers useful in the invention vary and are selected based on the disease indication being monitored and other factors known to the skilled artisan. Moreover, sensitivity and specificity may vary across biomarkers and that will influence biomarker selection. In some embodiments, the biomarker detected in a first sample is different than the biomarker detected in a second sample. In other preferred embodiments, the biomarkers detected across samples are of the same type. In embodiments, it is preferable to weight or quantify biomarkers in all samples taken in order to generate comparative analysis. Such comparative analysis may be indicative of the rate of progress of disease and its severity.

For example, the biomarker may comprise tumor cells, immune cells, bacterial cells, viral host cells, donor organ cells, microvascular cells, cell-free DNA, cell-free RNA, circulating tumor DNA, messenger RNA, miRNA, exosomes, proteins, hormones, and other analytes. The biomarker identified may depend on, for example, a specific patient, pathology, surgery type, and surgery site. By analyzing biomarkers in the obtained sample, methods of the invention may provide diagnostic or prognostic information. For example, by identifying circulating tumor cells or cell-free tumor DNA, cancer may be diagnosed in the subject.

As discussed above, in various aspects, biomarkers may be identified and quantified using methods known in the art. Suitable assays include, for example, nucleic acid sequencing, PCR, quantitative PCR, digital droplet PCR, Western blot target capture, proteomics, nucleic acid expression analysis, and antibody screening. For example, assays may include whole genome sequencing, next generation DNA sequencing, next generation RNA sequencing, multiplex PCR, methylation analysis, droplet PCR, droplet cell separation, or any combination thereof. Barcodes may be added to biomarkers as discussed above, and Unique molecular identifiers may also be provided to biomarkers in the sample.

For RNA or mRNA sequencing, sequencing may first comprise the step of preparing a cDNA library from barcoded RNA, for example through reverse transcription, and sequencing the cDNA. cDNA sequencing may advantageously allow for the quantification of gene expression within the single cell, and can be useful to identify characteristics of the single cell to, for example, make a diagnosis, prognosis, or determine drug effectiveness.

Also as discussed above, reverse transcription may be performed using without limitation dNTPs (mix of the nucleotides dATP, dCTP, dGTP and dTTP), buffer/s, detergent/s, or solvent/s, as required, and suitable enzyme such as polymerase or reverse transcriptase. Further, nucleic acid molecules may advantageously be amplified prior to sequencing. Amplification may comprise methods for creating copies of nucleic acids by using thermal cycling to expose reactants to repeated cycles of heating and cooling, and to permit different temperature-dependent reactions (e.g. by Polymerase chain reaction (PCR). Any suitable PCR method known in the art may be used in connection with the presently described methods. Non limiting examples of PCR reactions include real-time PCR, nested PCR, multiplex PCR, quantitative PCR, or touchdown PCR. Sequencing nucleic acid molecules may be performed by methods known in the art.

The method provides for evaluating the efficacy of treatment based on identifying a difference between a first sample collected and a second sample collected. In embodiments, the difference may be the presence or absence of biomarkers identified in the sample. In a non-limiting example, a patient may undergo a bowel resection surgery or colectomy to remove cancerous tumors, which results in a colostomy. The effluent collected in the colostomy bag is regarded as waste. However, according to methods of the invention, this effluent is rich in diagnostic material. Comparing biomarkers of a sample obtained at or near the time of surgery with biomarkers obtained from effluent post-treatment can be used to evaluate the efficacy of the treatment.

For example, in embodiments, efficacy of treatment or disease progression may be evaluated by identifying a difference in the amount of a circulating, cell-free biomarker such as cell-free DNA (cfDNA), cell-free tumor DNA (ctDNA) or cell-free RNA. The circulating, cell-free biomarker may also be extracellular vesicles, proteins, and metabolites from metastatic or normal organ physiologic turn over or impact of systemic drug treatment. For example, DNA methylation is an early event in cancer development that may be detected in circulating cell-free DNA. The information can be used for cancer diagnosis, prognosis, and monitoring.

ctDNA may indicate the presence of MRD following surgical resection and predict risk of recurrence with a high degree of precision. In embodiments, the sample is obtained at or near the time of the surgical procedure at a first time point. Further, the method contemplates obtaining a second sample at a second time point and identifying in the sample the presence or absence of the biomarker indicative of disease. In embodiments, the second sample is obtained from the patient after the patient has undergone treatment. Therefore, the presence or absence of the biomarker is an indication of treatment efficacy.

Thus, identifying a difference in the presence or absence of a biomarker related to the cancer removed from the patient by comparing biomarker(s) in samples obtained from effluent at first and second time points, may be indicative of minimal residual disease.

MRD can be defined as cancer persisting in a patient after treatment that may not be detected with current medical imaging modalities and is, therefore, an occult stage of cancer progression. Methods of the invention use biomarkers from effluent to evaluate MRD to see if the cancer treatment is working and to guide further treatment plans. In embodiments, methods of the invention use liquid biopsy approaches based on the detection of small numbers of circulating tumor cells (CTCs) or minute amounts of circulating cell-free tumor DNA (ctDNA) to detect MRD in patients with various malignancies. Using methods of the invention, monitoring CTCs and ctDNA during post-surgical follow-up assessments enables detection of disease relapse, or determines the success of surgical outcomes, earlier than is possible with current radiological imaging procedures. Further characterization of CTCs and ctDNA provides insights into the molecular evolution of MRD during tumor progression with implications for therapeutic interventions to delay or prevent metastatic relapse.

As noted above, methods of the invention may use utDNA, cfDNA, or ctDNA as biomarkers for disease recurrence, and/or determining the efficacy of treatment.

In another example, the measurement of biomarkers in peritoneal fluid such as cytokines or matrix metalloproteinases in the early post-operative period may be used to diagnose anastomotic leakage at a preclinical stage.

In embodiments, the biomarker may be selected from a nucleic acid, a protein, a bacterium, a cell, and a virus. As discussed above, the cell may be a cancer cell. Contemplated methods comprise identifying circulating tumor cells in lymphatic fluid at two or more time points; identifying cell-free tumor DNA in the sample of effluent at two or more time points; comparing a ratio of circulating tumor cells to cell-free DNA at the same two or more time points; and assessing the progression of disease or the efficacy of treatment as a changing ratio of cell-free tumor DNA to circulating tumor cells over the time interval of the two time points.

In other embodiments, the biomarker may be compared to an amount determined in blood or a lymph node. In embodiments, the biomarker comprises one or more of interleukin-1, interleukin-6, interleukin-10, a tumor necrosis factor, matrix metalloproteinase-1, matrix metalloproteinase-2, matrix metalloproteinase-9, matrix metalloproteinase-13, or a nucleic acid comprising a mutation. The method may further comprise the step of obtaining a genomic profile from the sample. For example, biomarkers for disease may be detected by sequencing, for example protein or nucleic acid sequencing. The biomarker may be DNA and/or RNA. Accordingly, methods of the present invention contemplate creating a genomic profile from DNA or RNA in the drain fluid. The profile may be a patient's germ line.

In alternative embodiments, the invention comprises methods for disease diagnosis in which a tumor cell or tumor-related nucleic acid is obtained from a surgical excision or biopsy. Then, a sample is taken from effluent to determine whether the same cancer biomarker is present in lymphatic fluid. In addition to a presence/absence test, the amount of the cancer biomarker in the effluent can be quantified and compared to a quantifiable amount of the cancer biomarker in a lymph node. Surgical procedures may include biopsy, excision, tumor resection, or other surgical interventions for treating, diagnosing, or staging disease. The invention takes advantage of the recognition that the lymphatic system is also involved in cancer progression, as entry of metastatic cancer cells into the lymphatic system can result in lymph node metastasis.

The invention may further comprise analyzing a blood sample for the same biomarker. In one embodiment, the cancer biomarker is identified in blood prior to identifying the cancer biomarker in the effluent sample.

A sample of the effluent may be obtained by any known method. For example, the sample may be obtained using a catheter or a drain port and may be actively or passively collected. The sample may be collected in or transferred to a container, for example a sample vessel, such as a vial, flask, or ampule, suitable for the collection of medical specimens.

Samples may be obtained at any time during or following an interventional procedure. For example, drain fluid may be collected at the time of intervention and then periodically over the course of hours, days or weeks.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for monitoring efficacy of a treatment, the method comprising the steps of:
obtaining, as a first sample, effluent from a patient having undergone a surgical procedure, wherein the first sample is obtained at a first time point;
obtaining, as a second sample, effluent from the patient, wherein the second sample is obtained at a second time point;
identifying in the sample a difference in the first sample and the second sample in a biomarker indicative of disease; and
determining the efficacy of treatment based on the presence or absence of the biomarker in the sample.

2. The method of claim 1, wherein the first sample is obtained at or near the time of the surgical procedure.

3. The method of claim 2, the difference identified is the presence or absence of the biomarker indicative of disease, wherein the presence or absence of the biomarker is an indication of treatment efficacy.

4. The method of claim 3, wherein the second sample is obtained after the patient has undergone treatment.

5. The method of claim 1, wherein the first sample and/or the second samples are obtained from a bag or pouch system connected to connected to a stoma created by an ostomy.

6. The method of claim 5, wherein the first sample and/or the second sample are collected from a colostomy, ileostomy, or urostomy bag.

7. The method of claim 1, wherein the first sample and/or the second sample is collected from a percutaneous catheter, peritoneal port, or intraperitoneal drain.

8. The method of claim 1, wherein the presence of the biomarker is indicative of minimal residual disease.

9. The method of claim 1, wherein the biomarker is selected from a nucleic acid, a protein, a bacterium, a cell, and a virus.

10. The method of claim 1, wherein the biomarker is a ratio of circulating tumor cells to cell-free DNA.

11. The method of claim 1, wherein the biomarker comprises one or more of interleukin-1, interleukin-6, interleukin-10, a tumor necrosis factor, matrix metalloproteinase-1, matrix metalloproteinase-2, matrix metalloproteinase-9, matrix metalloproteinase-13, or a nucleic acid comprising a mutation.

12. A method for assessing surgical success, the method comprising the steps of:
obtaining, as a sample, effluent from a patient having undergone a surgical procedure;
identifying in the sample a biomarker indicative of disease; and
determining the success of the surgery based on the presence or absence of the biomarker in the sample.

13. The method of claim 12, wherein the sample is obtained from a bag or pouch system connected to a stoma created by an ostomy.

14. The method of claim 13, wherein the sample is collected from a colostomy, ileostomy, jejunostomy, duodostomy, gastrostomy, or urostomy bag.

15. The method of claim 12, wherein the sample is collected from a percutaneous catheter, peritoneal port, or intraperitoneal drain.

16. The method of claim 12, wherein the presence of the biomarker is indicative of minimal residual disease.

17. The method of claim 12, wherein the biomarker is selected from a nucleic acid, a protein, a bacterium, a cell, and a virus.

18. The method of claim 12, wherein the biomarker is a ratio of circulating tumor cells to cell-free DNA.

19. The method of claim 12, wherein the biomarker comprises one or more of interleukin-1, interleukin-6, interleukin-10, a tumor necrosis factor, matrix metalloproteinase-1, matrix metalloproteinase-2, matrix metalloproteinase-9, matrix metalloproteinase-13, or a nucleic acid comprising a mutation.

20. The method of claim 12, further comprising the step of obtaining a genomic profile from the sample.
